(19) United States
(12) Patent Application Publication
(10) Pub. No.: US 2006/0034823 A1
(43) Pub. Date: Feb. 16, 2006

(54) Method of production and use of crotoxin as an analgesic

(76) Inventors: Paul Reid, Ft. Lauderdale, FL (US); Zheng Hong Qin, Malden, MA (US)

Correspondence Address: ROBERT J. VAN DER WALL;GABLES ONE TOWER, 1320 SOUTH DIXIE HIGHWAY, SUITE 1275, CORAL GABLES, FL, 33146, US

(21) Appl. No.: 10/917,143

(22) Filed: Aug. 13, 2004

(51) Int. Cl. A61K 38/48; A61K 31/60

(52) U.S. Cl. 424/94.63; 514/165

(57) ABSTRACT

Disclosed a pharmaceutical composition including one of crotoxin, mojavetoxin or a related toxin and a carrier for use in the treatment of chronic pain, especially to the treatment of heretofore intractable pain such as that associated with advanced cancer wherein the treatment is independent of the reduction of tumors. The crotoxin is preferably obtained from the snake Crotalus durissus terrificus and the mojavetoxin is obtained from the rattlesnake Crotalus scutulatus scutulatus. Preferably the composition further comprises an effective amount of acetylsalicylic acid whereby the toxin and acetylsalicylic acid together produce a synergistic effect providing enhanced pain relief. The composition of the invention may be delivered in parenteral (i.p.) (intravenous, intramuscular or subcutaneous) applications. Alternatively, the composition may be applied as a topical application. The biological activity of the composition is characterized by its ability to bind to presynaptic and postsynapic receptor sites. This results in an inhibition of aceylcholine release and activity.

### BACKGROUND OF THE INVENTION

1. Field of the Invention

The present invention relates to a class of proteins, a process of production thereof, and a method for the treatment of chronic pain, especially to the treatment of heretofore intractable pain such as that associated with advanced cancer. The pain associated with neurological conditions, rheumatoid arthritis, viral infections and lesions may also respond to treatment with the composition of the present invention. The composition consists of a beta-neurotoxin, optionally in admixture with pharmaceutically acceptable excipients or carriers for either parenteral or topical administration. The method of use includes administering a beta-neurotoxin that is characterized by its ability to bind to presynaptic receptors resulting in an inhibition of acetylcholine release. In this specification, the term “base” means a pharmaceutically acceptable carrier, for example, ointment, creme, lotion or the like.

2. Description of the Prior Art

Research into the pharmacological properties of natural products led to the identification of many compounds with a potent biological activity, which can result in clinically useful therapeutic agents (Verdine, 1996). Plant, microbial and animal toxins are of particular interest, due to their strong pharmacological activity and their selectivity for a particular site of action. They can be employed directly as therapeutic agents or can serve as starting points for drug design (Harvey et al., 1998).

The frequent and severe side effects accompanying chemotherapy conventionally employed for treatment of tumors has stimulated the search for new compounds, able to counteract the uncontrolled proliferation of tumor cells by cytostatic or cytolytic mechanisms. This search was also directed towards natural products following different research lines. One research line screened substances for cytotoxic activity, and yielded a large variety of toxic compounds, like arabinose nucleosides, plant products (vinblastin, vincristine, taxol); tunicates peptides (dolostatin 10, didemnin B), macrocyclic lactames (bryostatin 1), etc. Another research line used large molecular weight toxic proteins from plants (ricin, abrin) or microorganisms (diphtheria toxin, Pseudomonas aeruginosa exotoxin) with known killing mechanisms. However, these are extremely toxic products which do not have any specificity, therefore they cannot be used directly. They were used as immunotoxins, i.e. genetic engineered fusion proteins with monoclonal antibodies directed against tumor antigens.

Snake venoms also possess cytotoxic activity on tumor cell lines in-vitro, although their anticancer properties in animal models have been studied only in a limited manner. (Stocker, 1990). The venoms from cobras (Gen. Naja and Hemachatus) contain large amounts of basic, non enzymatic peptides of molecular weight 6.8 kD referred to generically as cardiotoxins, (Harvey, 1985, 1991) which display cytolytic activity on a broad variety of tumor as well as normal cells. Indeed, the observation that cytolytic activity of whole Naja nigricollis venom was higher than that expected from the amount of cardiotoxin present in the venom led to the isolation of a cytotoxic phospholipase A₂ (nigexin, Chwetzoff, 1988).

Phospholipases A₂ are conspicuous components of snake venoms. These enzymes are compact globular proteins with molecular weight of about 14000 kD as monomers or are found as multimeric complexes. They exhibit a high degree of similarity in amino acid sequences, secondary and tertiary structure (Verheij et al., 1981). They catalyze the stereospecific, Ca²⁺-dependent hydrolysis of the fatty acyl ester bond in position 2 of all common 3-sn-phosphoglycerides (plasmalogens or glyceryl ethers) resulting in free fatty acid and the 1-acyl (1-alkenyl or 1-alkyl) phosphoglyceride or lysophospholipid (van Deenen & De Haas, 1963). At difference with the highly conserved (invariant) amino acid residues responsible for their catalytic activity (i.e., the catalytic network), the structural elements responsible for the wide variety of pharmacological effects exhibited by this large family of homologous proteins are presently unknown (Kini and Evans, 1989; Kini, 1997). In spite of their similarities in sequence, secondary and tertiary structures, these enzymes may induce one or more pharmacological effects (i.e., neurotoxic, myotoxic, cardiotoxic, anticoagulant, hemolytic, edema-inducing activities, etc.), but not all the effects are exhibited by all phospholipases A₂.

Crotoxin, the neurotoxic phospholipase A₂ from Crotalus durissus terrificus (South American rattlesnake) venom displays cytotoxical activity in vitro against a number of murine and human tumor cell lines. The cytocidal effect of the unmodified toxin exhibited some degree of selectivity towards some tumor cell lines, since after 8 h incubation with 10 μg/ml crotoxin, all the Hs87T (breast ductal carcinoma, human) and Lu-1 (lung adenocarcinoma, human) cells were killed while 20% of 3T3-fibroblasts and 45% of normal human keratinocytes remained viable after 72 hours. The toxin of the Mojave rattlesnake Crotalus scutulatus scutulatus, namely mojavetoxin, has a very high degree of homology with that of the South American rattlesnake and has the same activity as described herein for crotoxin. Accordingly, while the present disclosure refers particularly to crotoxin, it is to be understood that it is also intended to encompass mojavetoxin and other homologous related toxins from similar species.

Although not fully understood, it is believed that this anticancer mechanism involves the recognition by crotoxin of a set of structural elements present in the membrane of the target cells, which results in binding of the toxin. Structural perturbation of the membrane resulting from anchoring of the toxin and subsequent hydrolysis of membrane phospholipids leads to cell death. Thus, crotoxin differs from other cytotoxic agents in that:

1. It appears to be a self-targeted toxin (through the A subunit); with regard to targeting crotoxin is a bi-partie toxin consisting of two distinct units, A and B. A is also known as crotapotin and B is Phospholipase A. Crotapotin dictates the cell that will be affected by the toxin thereby targeting it. Crotapotin recognizes certain surface markers that allow it to attach and release the phospholipases that causes the cell damage.

The cell surface markers have been identified as calcium channels, biogenic amine transporters and, for cancer cells, the epidermal growth factor (EGF). The surface target could be one of them or all three. Without crotapotin the phopholipase is indiscriminate in it's destruction of cells. Usefully, the crotapotin target is highly expressed on tumor cells thereby resulting in little collateral damage.

2. It exerts its cytotoxic activity on the cell membrane of the target cells; and

3. It is not an enzyme inhibitor, an antimetabolite and does not interact with DNA.

The possibility of achieving cytotoxicity in malignant cells by means of the specific binding of a phospholipase A₂ and subsequent phospholipid hydrolysis constitutes a novel approach to cancer therapy. This cytotoxic activity of crotoxin is distinct from the protein's neurotoxic capacity.

Prior art patent publications include the following United States patents: U.S. Pat. No. 4,341,762 (Haast) which discloses use of snake venoms for treatment of neurological and related disorders. Other U.S. patents relating to complexes as cytotoxins agent include: U.S. Pat. No. 5,164,196 (Plata, et al), U.S. Pat. No. 5,232,911(Vidal), U.S. Pat. Nos. 5,565,431 and 6,307,031 (Lipps).

### SUMMARY OF THE INVENTION

Bearing in mind the foregoing, it is a principal object of the invention to provide a composition and method for treating pain associated with advanced cancer and other chronic pain conditions, such as neuropathy, painful viral infections and their lesions in addition to rheumatic pain.

It is a further object of the invention to provide a composition and therapy for the treatment of pain of the aforementioned type, which composition and therapy are safe, effective and may be administered over long periods of time.

Other objects and advantages will be apparent to those skilled in the art from the following descriptions and appended claims.

The present invention accomplishes the above-stated objectives, as well as others, as may be determined by a fair reading and interpretation of the entire specification.

Accordingly, the present invention provides a pharmaceutical composition having pharmacological activity comprising a therapeutically effective amount of crotoxin, mojavetoxin or a related toxin having corresponding biological activity and a pharmaceutically acceptable carrier for use in inhibiting or controlling pain.

In accordance with a primary aspect of the the invention, the crotoxin is preferably obtained from the snake Crotalus durissus terrificus. Mojavetoxin is obtainable from the rattlesnake Crotalus scutulatus scutulatus. Preferably the composition further comprises an effective amount of acetylsalicylic acid whereby the toxin and acetylsalicylic acid together produce a synergistic effect providing enhanced pain relief.

In accordance with a secondary aspect of the invention, the composition of the invention may be delivered parenterally (intravenous, intramuscular or subcutaneous). A toxin composition administered parenterally delivering between 0.13 mcg.kg⁻¹ of body weight per day up to a maximum of 40 mcg.kg⁻¹ of body weight per day (75 Kg being employed as the average human weight). Preferably the toxin is crotoxin. Alternatively, the composition may be applied as a topical application in which case the concentration of toxin ranges from 6 mcg per gram of base up to 1 mg per gram of base. Alternately, for topical application the whole venom may be employed so that the crotoxin need not be purified providing cost savings from the venom. Preferably, the average crotoxin concentration in the composition is 100-200 mcg per gram of base.

In accordance with a tertiary aspect of the invention, there is provided a method of producing analgesia and/or enhancing analgesia comprising administering an effective amount of crotoxin, mojavetoxin or a related toxin having corresponding biological activity that is characterized by its ability to bind to presynaptic receptor sites. This results in an inhibition of aceylcholine release.

The method of treatment of pain in the human or animal body comprises administering to the human or animal, a pharmaceutically effective amount of a composition comprising the toxin, ideally crotoxin. Advantageously, the method involves use of a composition including a therapeutically effective amount of acetylsalicylic acid whereby the toxin (preferably crotoxin) and acetylsalicylic acid together in an admixture produce a synergistic effect providing enhanced pain relief. Preferably, the method of treatment of pain comprises regularly administering the crotoxin composition ranging from at least once every other day to several applications daily.

### DETAILED DESCRIPTION OF THE PREFERRED EMBODIMENTS

Detailed embodiments of the present invention are disclosed herein; however, it is to be understood that the disclosed embodiments are merely exemplary of the invention which may be embodied in various forms. Therefore, specific functional details disclosed herein are not to be interpreted as limiting, but merely as a basis for the appended claims and as a representative basis for teaching one skilled in the art to variously employ the present invention in virtually any appropriate circumstance.

Chronic or intractable pain, such as may occur in degenerative bone diseases and cancer, is a debilitating condition which is treated with a variety of analgesic agents, and often opioid compounds, such as morphine. In general, brain pathways governing the perception of pain are still incompletely understood. Sensory afferent synaptic connections to the spinal cord, termed “nociceptive pathways” have been documented in some detail. In the first leg of such pathways, C- and A-fibers, which project from peripheral sites to the spinal cord, carry nociceptive signals. Polysynaptic junctions in the dorsal horn of the spinal cord are involved in the relay and modulation of sensations of pain to various regions of the brain, including the periaqueductal grey region (McGeer). Analgesia, or the reduction of pain perception, can be effected directly by decreasing transmission along such nociceptive pathways. Analgesic opiates are thought to act by mimicking the effects of endorphin or enkephalin peptide-containing neurons, which synapse pre-synaptically at the C- or A-fiber terminal and which, when they fire, inhibit release of glutamate and substance P. The key transmitter is glutamate that activates N-methyl-d-aspartate (NMDA) and non-NMDA receptors on spinal cord neurons. Substance P (SP) is a neuropeptide which is abundant in the periphery and the central nervous system, where it is colocalized with other neurotransmitters such as serotonin or dopamine. SP has been proposed to play a role in the regulation of pain including migraine and fibromyalgia, asthma, inflammatory bowel disease, emesis, and psoriasis as well as in central nervous system disorders.

The synthesis of analgesics, particularly of morphine-like compounds, has always been a point of major interest in drug research. For decades, scientists throughout the world have attempted to develop effective analgesics by “re-building” the morphine molecule, considering its constitution a combination of certain “basic skeletons” from which they started their syntheses. Meperidine hydrochloride (also known as Dolantin or Demerol) is one such synthetic narcotic analgesic. It is one-tenth as potent an analgesic as morphine and its analgesic effect is halved again when given orally rather than parenterally. The onset of activity occurs within 10-45 minutes with a duration of 2-4 hours. It has superceded morphine as the preferred analgesic for moderate to severe pain. It has been found to be particularly useful for minor surgery, as in orthopedics, ophthalmology, rhinology, laryngology, and dentistry. It is also used in parenteral form for preoperative medication, adjunct to anesthesia and obstetrical analgesia. Like morphine, its binding to opioid receptors produces both psychologic and physical dependence with overdosing causing severe respiratory depression in addition to a number of other undesirable side effects and drug interactions.

Although calcium blocking agents, including a number of L-type calcium channel antagonists, have been tested as adjunct therapy to morphine analgesia, positive results are attributed to direct effects on calcium availability, since calcium itself is known to attenuate the analgesic effects of certain opioid compounds (Ben-Sreti). Ethylene glycol-bis(2-aminoethylether)-N,N,N″,N′-tetraacetic acid (EGTA), a calcium chelating agent, is effective in increasing the analgesic effects of opioids. Moreover, in some cases, results from tests of calcium antagonists as adjunct therapy to opioids have been contradictory; some L-type calcium channel antagonists have been shown to increase the effects of opioids, while others of these compounds have been shown to decrease opioid effects (Contreras).

Due to the limitations of such analgesics, a number of novel alternatives are currently under investigation, including neuronal nicotinic acetylcholine receptor (NAChR) agonists. Acute administration of nicotine induces analgesia with subsequent development of tolerance. Interestingly, in nicotine-naive rats, injection of the nicotinic receptor antagonist mecamylamine into the nucleus accumbens (where the site for activity of substances of abuse such as opioids has been implicated in pain modulation) blocked antinociception produced by either systemic morphine, intra-accumbens co-administration of a mu- and a delta-opioid receptor agonist, or noxious stimulation (i.e., subdermal capsaicin in the hindpaw). Intra-accumbens mecamylamine by itself precipitated significant hyperalgesia in nicotine-tolerant rats which could be suppressed by noxious stimulation as well as by morphine. Thus, nicotinic receptors have been found to play a role in modulating pain transmission in the central nervous system (CNS). Activation of other cholinergic pathways by nicotine and nicotinic agonists has been shown to elicit antinociceptive effects in a variety of species and pain tests.

The analgesic effect of snake venom proteins has been known since antiquity and several authors have pointed out the efficiency of the administration of crude cobra or rattlesnake venoms in the treatment of trigeminal neuralgias, tabetic and pain caused by tumors. In the cases of tumoral pains, the patients could be maintained without the administration of morphine in 70% of the cases. Obviously, at the time, crude venoms were employed without even an adequate knowledge of the source or mechanism. Sometimes venoms from cobras captured in India or South Africa were employed indistinctly (see, Haast, U.S. Pat. No. 4,341,762). A number of venom-derived neurotoxins such as conotoxin SNX111, crotamine and cobrotoxin have demonstrated antinociceptive properties. With respect to crotamine, it was assumed that this represented the anti-nociceptive component of Crotalus durissus terrificus venom. However, our studies with crotamine in animal models of pain have failed to confirm these published observations.

Crotoxin as a treatment for malignancies has been previously described and the reduction in pain was assumed to be due to the amelioration of the tumor mass. The use of crotoxin solely to relieve pain has not been described heretofore. Accordingly, the present invention relates to a newly described activity of crotoxin.

Crotoxin, nigexine and taipoxin are neurotoxic phospholipases A2 capable of affecting the presynaptic activity to bring about the ultimate blockade of synaptic transmission. Crotoxin impairs the release of acetylcholine at neuromuscular junctions, primarily at the presynaptic level. It also serves to inactivate nicotinic receptors rendering them non-responsive to acetylcholine. The enzymatic activity of crotoxin has generally been considered to be necessary but not sufficient for the blockade. Since many phospholipases A2 with comparable or even higher enzymatic activity are not neurotoxic, it has been postulated that the difference lies in the affinity of binding to the presynaptic membrane.

Crotoxin is a non covalent complex having a molecular weight of 24 kD and is formed by two non identical subunits: a basic one (crotoxin subunit B, molecular weight 14.5 kD) and an acidic one (crotoxin subunit A, molecular weight 9.5 kD). Crotoxin subunit A is non-toxic and devoid of catalytic activity. It is formed by three polypeptide chains A, B and C cross-linked by seven disulfide bonds. When properly aligned, the polypeptide chains (A, B and C) exhibit sequence similarities with other non-toxic phospholipases. Isoforms of subunit A which differ in two or three amino acid residues at the beginning and at the end of chain A appear to be generated by the proteolytic cleavage of a precursor polypeptide homologous to a phospholipase A₂ (Bon, 1997). The structure and production is described in detail in the specifications of the following patents: U.S. Pat. No. 5,164,196 and U.S. Pat. No. 5,232,911.

It has been previously shown that iodinated crotoxin and taipoxin bind specifically with high affinity to the isolated synaptic membrane fraction from guinea-pig brain, whereas specific binding is not detected with the nontoxic pancreatic phospholipase A2. Experiments based on photoaffinity labeling and simple chemical cross-linking techniques have led to the identification of three polypeptides preferentially present in neuronal membranes as (subunits of) the binding protein(s) for crotoxin and/or taipoxin. Some, but not all, other toxic phospholipases A2 also appear to be ligands for the three polypeptides. It has been found that under Ca(2+)-free condition, taipoxin or crotoxin inhibit with a 50% inhibition constant (IC50) of 20-1000 nM the Na(+)-dependent uptake of norepinephrine, dopamine and serotonin by the synaptosomes. In contrast, choline uptake is not affected.

Crotoxin is known to desensitize the nicotinic receptor of Torpedo marmorata and Electrophorus electricus electroplaques. It has been found that the purely cholinergic synaptosomes from the Torpedo electric organ provided a convenient model to study the pharmacology of crotoxin and other related neurotoxins [Delot, E., & Bon, C. (1992) J. Neurochem. 58, 311-319]. Labeled 125I crotoxin demonstrated saturable binding to Torpedo presynaptic membranes. In the range of concentrations that was effective on synaptosomes, crotoxin bound to a single class of sites without cooperativity. 4-Aminopyridine antagonism of the crotoxin-induced blockade of the end-plate depolarization produced by carbachol show that the postsynaptic effect of crotoxin at the guinea-pig muscle end-plate also results from nicotinic receptor desensitization. Therefore, the purported dual mechanism of crotoxin suggests that the anti-nociceptive effect could result from the impairment of acetylcholine release from the presynaptic surface and nicotinic acetylcholine receptor desensitization. Desensitization of the nicotinic receptor would mimic the analgesic effects described for nicotine. While nicotine is an agonist of its respective receptor, it is the resulting desensitization of the receptor that is now believed to form the basis of its antinociceptive effects.

The present invention will now be more particularly described with reference to examples which describe various embodiments of the invention with particular reference to crotoxin. However, very similar toxins such as mojavetoxin, which has a high degree of homology with crotoxin and which has very similar biological activity, are to be understood to lie within the scope of the invention.

The production of purified crotoxin is disclosed in U.S. Pat. No. 5,164,196 (Plata, et al.), and U.S. Pat. No. 5,232,911 (Vidal). The resulting neurotoxin solution, i.e., crotoxin solution, is filter sterilized to remove residual bacteria and examined for appropriate levels of endotoxin. The solution needs to be diluted usually to between 500 and 2000 mcg/ml prior to filling and administration (1 mg/ml crotoxin approximates to 1.7 optical density units at A280 nm). Any suitable preservative for parenteral administration can be employed such as methyl paraben, benzalkonium chloride or metacreosol.

In the treatment of pain, the administration of crotoxin is required regularly, at least once every other day extending to several applications daily. Parenteral (intravenous, intramuscular or subcutaneous) neurotoxin composition delivering between 0.13 mcg.kg⁻¹ of body weight per day up to a maximum of 40 mcg.kg⁻¹ of body weight per day (75 Kg being employed as the average human weight). Studies have shown the average dose to be between 1.33 and 13.33 mcg.kg¹ per day for purified neurotoxin preparations. Higher doses can be employed for more rapid onset of effect with the preferred route being intravenous. In some cases, patients may experience injection site and immune reactions which can be reduced by tolerizing the individual to the drug through the injection of low doses over a period of time. A two-week protocol of less than 1.33 mcg.kg⁻¹ i.m. per day permits the immune system to acclimatize to the drug prior to initiating higher therapeutic dose schedules.

The standard formulation of crotoxin for parenteral administration is:

1. Crotoxin, at a concentration of 500-2000 micrograms (mcg) per mL (0.05-0.2%) although it can be as low as 90 mcg (0.009%).

2. The carrier solution chloride (0.9%) suitable for injection.

3. The preferred preservative is benzalkonium chloride at a concentration of 0.001% any other preservative can be employed that will meet United States Pharmacopea (USP) challenge requirements.

For topical applications, the concentration of the present neurotoxin range from a minimum of 6 mcg per gram of base up to 1 mg per gram of base (lotion or creme). The applicable topical concentrations of venom are 2-3 fold greater than that for the purified neurotoxin as the neurotoxin accounts for approximately 40% of the composition of the venom. The raw venom should be collected in depyrogenated glassware (to minimize endotoxin contamination) and filtered sterilized prior to formulation as the venom can contain pathogenic organisms such as Clostridium perfringens (the causal agent of gas gangrene). Viral contaminants in the venom are not considered significant due to their limited host range. Furthermore, the use of benzalkonium chloride as a preservative is a known antiviral agent in addition to a penetration enhancer. The average crotoxin concentration of 100-200 mcg per gram of base is preferable. The rate of application can range from an infrequent, as needed basis, to several applications per day particularly where the application is for the control of pain. The treatment of a condition like shingles may require 4 to 5 topical applications per day in order to reduce pain and speed healing.

In order to verify the antinociceptive properties of Crotoxin, a number of tests were conducted in three accepted animal models of pain: the hot-plate test, the acetic acid writhing test and the tail-flick test. Analgesia is conveniently measured in these animal models, in which an animal's response to a given pain stimulus is measured.

In the hot-plate test, a animal is positioned on a hot plate and is exposed to a standard heat source, and the time that the animal voluntarily endures the heat, prior to licking its paws, is recorded. In the acid-writhing test, the animal is injected into the intraperitoneal cavity and results in a writhing response. For the tail-flick test the animal's tail is subjected to heat or electrical stimulus and the time taken for the animal to remove its tail away from the noxious stimulus is recorded. Analgesics, particularly opioid analgesics, will prolong these times relative to controls.

Naloxone hydrochloride was purchased from Sigma. Male and female Kunming mice weighing 18-22 g and Sprague-Dawley ♀♂rats weighing 200-250 g were purchased from the Medical Experimental Animal Center, Soochow University, Suzhou, P. R. China (Grade 2, Certification No. 98018). Data analysis: The data were expressed as x±s. Statistical significance of differences was determined by one-way analysis of variance (ANOVA).

### EXAMPLE 1

Purification of Crotoxin

Crotoxin was purified from venom through size exclusion chromatography. A Sephadex G75 (Amersham Biosciences) column with nominal dimensions of 2.5 cm by 100 cm was set up using 0.9% sterile saline as the suspension buffer. Saline for irrigation or injection makes an excellent suspension buffer. The venom (usually 100-200 mg) was dissolved overnight in the suspension buffer at 10-20 mg/ml. It was then filtered and loaded onto the column. Usually 4 peaks are obtained when monitoring at 280 nm and the second peak represents crotoxin. In this method the material in peak 2 can be employed directly from the column. If the material is to be lyophilized, it is necessary to dialyse the collected fractions against sterile water prior to freeze drying. The average yield of crotoxin from 100 mg of venom is approximately 30 mg.

### EXAMPLE 2

Crotoxin Assay in Competition with Dolantin in Mouse Hot-Plate Assay.

Female mice were placed on a hot plate with temperature set up to 55±5° C. The latency time for mice to lick hind paw was recorded as pain threshold. The baseline pain threshold was obtained by averaging values of 2 measurements before drug administration. Mice with 5-20 seconds pain threshold were used in the experiments. After drug administration, pain threshold was determined by the same method and a cutoff time of 60 seconds was used to minimize potential hazardous effects. Crotoxin: 45-90 μg/kg was effective. The action lasted over 180 min. Small dose (32 μg/kg), medium dose (45 μg/kg) and large dose (90 μg/kg) levels were administered to separate groups of test mice (sample size 10). Toxic effects (body shaking) were observed at the large dose of 90 μg/kg resulting in the use of reduced crotoxin levels in the large dose (66.5 μg/kg) in subsequent experiments. The results are presented in Table 1.

### EXAMPLE 3

Hot Plate Test in Mice (Repeat Assay in Comparison to Acetylsalicylic Acid):

Crotoxin was administered at dose levels of 29.5, 44.3, or 66.5 mcg.kg^{-1 -1} (ip) indicated in Table 2 as small, medium and large doses, respectively. Crotoxin exhibited a dose-dependent prolonging of the latency time of the mouse in response to pain stimulation induced by heat. The analgesic effects of crotoxin appeared at 1 hour, and the most intense analgesic activity was seen at 3 hours after drug administration. The half effective dose (ED₅₀) of the antinociceptive effects of crotoxin was 53.7(42.55±67.77, 95% confidence limit) mg/kg⁻¹. The results are shown in Table 2.

### EXAMPLE 4

Effects of Crotoxin on the Acetic Acid Writhing Response in Mice:

The mice were randomly divided into 5 groups (n=10 in each group): saline control, acetylsalicylic acid 400 mg/kg⁻¹, crotoxin 66.5, 44.3, or 29.5 mcg.kg^{-1 -1}. Pain threshold was measured 3 h after drug administration. Crotoxin 29.5, 44.3, or 66.5 mg/kg⁻¹ (ip) elicited a dose-dependent inhibition of writhing response. The ED₅₀ of the antinociceptive effects of crotoxin was 39.02 (28.59±53.20, 95% confidence limit) mcg.kg^{-1 -1}. The results are shown in Table 3.

### EXAMPLE 5

Tail Flick Assay in Rat

Rats were kept in special-made holders for tail-flick test. Pain threshold was measured with WQ-9E Pain Threshold meter. The steadily increasing current was applied to rat tail as noxious stimulus via an electrode connected to the meter. The smallest intensity of the current (mA) provoking the tail flick was recorded as pain threshold. Rats with pain threshold<0.3 mA were used in experiment. Pain threshold was measured 2 times before administration and the average value was used as baseline pain threshold. After administration, pain threshold was determined by the same method and a cutoff current of 1 mA was used to minimize potential hazardous effects. In comparison with control group, crotoxin 44.3 mcg.kg^{-1 -1} significantly inhibited the tail flick reflex in rats. The analgesic effects of crotoxin reached the peak at 2-3 hours after drug administration. The results are shown in Table 4.

### EXAMPLE 6

Analgesic Action of icv Injection of Crotoxin in the Mouse Acetic Acid Writhing Test

The mice were randomly divided into 2 groups (n=10 in each group): saline control and crotoxin 0.3 mcg.kg^{-1 -1}. The effects of crotoxin on pain threshold were measured 1 hour after crotoxin or saline administration. In the acetic acid writhing test, the number of writhings after crotoxin (icv) 0.3 cg/kg⁻¹ was significantly less than that in saline group, indicating that icv injection of crotoxin had marked analgesic effects. The results are shown in Table 5.

### EXAMPLE 7

Effects of Atropine on Analgesia Induced by Crotoxin Hot Plate Test

The mice were randomly divided into 4 groups (n=10 in each group): saline control, crotoxin 44.3 mcg.kg^{-1 -1}, atropine (Atr) 0.5 mg/kg⁻¹, and crotoxin+atropine. Atropine 0.5 mg/kg⁻¹ or NS (im) was administrated 1.5 h after crotoxin. Pain threshold was determined 1.5 h after atropine administration. In the hot plate test and the acetic acid writhing test in mice, atropine 0.5 mg/kg⁻¹ (im) and atropine 10 mg/kg⁻¹ (ip) had no significant effect on the pain threshold. Both crotoxin and crotoxin combined with atropine exhibited marked analgesia. There was no significant difference between two groups. The results are shown in Table 6.

Acetic acid writhing test: The mice were randomly divided into 4 groups (n=10 in each group): saline control, crotoxin 44.3 mcg.kg^{-1 -1}, atropine 10 mg/kg⁻¹, and crotoxin+atropine. Atropine 10 mg/kg⁻¹ or NS (i.p.) was administrated 1.5 hours after crotoxin. Pain threshold was determined 1.5 hours after atropine administration. The results are shown in Table 7.

### EXAMPLE 8

Effects of Naloxone on Analgesia Induced by Crotoxin in Mouse Hot Plate Test

The mice were randomly divided into 4 groups (n=10 in each group): saline control, crotoxin 44.3 mcg.kg^{-1 -1}, naloxone (Nal) 3 mg/kg⁻¹, and crotoxin+naloxone. Naloxone 3 mg/kg⁻¹ or NS (i.p.) was administrated 2.5 hours after crotoxin. Pain threshold was determined 30 minutes after naloxone administration. In the hot plate test, naloxone 3 mg/kg⁻¹ (ip) had no significant influence on the pain threshold. Both crotoxin 44.3 mcg.kg^{-1 -1} and crotoxin combined with naloxone produced marked analgesia. There was no significant difference between the two groups. The results are shown in Table 8.

### EXAMPLE 9

Effects of Acetylsalicylic Acid on Analgesia of Crotoxin in the Mouse Acetic Acid Writhing Test

The mice were randomly divided into 4 groups (n=10 in each group): saline control, crotoxin 44.3 mcg.kg^{-1 -1}, acetylsalicylic acid (Ace) 300 mg/kg⁻¹, and crotoxin+acetylsalicylic acid. Acetylsalicylic acid 300 mg/kg⁻¹ (im) and crotoxin 44.3 mcg.kg^{-1 -1} (im) were administrated at the same time. Pain threshold was determined 2 h after crotoxin administration. In the mouse acetic acid test, acetylsalicylic acid 300 mg/kg⁻¹ and crotoxin 44.3 mcg.kg^{-1 -1} produced significant analgesic effects. The analgesia of crotoxin combined with acetylsalicylic acid was stronger than that of either crotoxin or acetylsalicylic acid alone. There was a significant difference between two groups. The results are shown in Table 9.

From the animal studies three attributes could be ascribed to Crotoxin with respect to its analgesic activity:

(1) Crotoxin 66.5, 44.3, 29.5 mcg.kg⁻¹ (ip) exhibited a dose-dependent analgesic activity in both hot plate test and acetic acid writhing test in mice. The peak effect of its analgesia was seen 3 hours after administration of crotoxin (ip). The ED₅₀ of its analgesia was 53.70 mcg.kg⁻¹ (42.55±67.77, 95% confidence limit) in the hot plate test, or 39.02 mcg.kg⁻¹ (28.59±53.20, 95% confidence limit) in the acetic acid writhing test. Crotoxin 44.3 mcg.kg⁻¹ (ip) had significant analgesic actions in the rat tail-flick test induced by electric stimulation. In the mice acetic acid writhing test, icv injection of crotoxin at the dose of 1/130 of systemic effective dose (0.3 mg.kg⁻¹) produced marked analgesic effects.

(2) Atropine 0.5 mg.kg⁻¹ (im) or 10 mg.kg⁻¹ (ip) failed to antagonize the analgesic effects of crotoxin (44.3 μg.kg⁻¹) in the hot plate test and acetic acid writhing test in mice. In the mice hot plate test, naloxone mg.kg⁻¹ (ip) had no effect on the analgesia induced by crotoxin.

(3) In the mice acetic acid-writhing test, acetylsalicylic acid 300 mg.kg⁻¹ (ip) significantly enhanced the analgesic effects of crotoxin.

The results clearly demonstrate that Crotoxin has analgesic effects. Furthermore the results show that crotoxin has analgesic effects in the absence of cancer or other chronic pain conditions. The results suggest that the site of analgesic actions of crotoxin may be mainly mediated by the central nervous system. The central cholinergic system and the central endogenous opioid peptidergic system appear not to be involved in antinociceptive actions of crotoxin. The combination of acetylsalicylic acid with crotoxin can increase the analgesic effects of crotoxin, suggesting that crotoxin may also produce peripheral analgesia similar to acetylsalicylic acid in addition to its central actions.

Crotoxin was compared to Dolantin and Acetylsalicylic acid for its ability to delay the hot-plate response in the hot-plate model (see Examples 1 and 2). In comparison to the control animals, animals treated with 40 mg.kg⁻¹ Dolantin had a rapid effect on the animals, showing a delay in the licking response from 13.2 seconds at time zero (T₀) to its maximal effect of 56 seconds at 30 minutes. At 90 minutes, the effect of Dolantin was wearing off, showing a 36 second delay in the licking response reducing further to 34 seconds at 120 minutes. The effect of crotoxin (90 mcg.kg⁻¹ administered i.p.) was slower to onset, though ultimately it achieved an almost equivalent effect to Dolantin. At T₉₀, the tail flick response was 25 seconds. The drug's effect continued to increase with time maximizing at 45 seconds at 180 minutes (the test's end point). The data suggested that crotoxin had an activity equivalent in potency to Dolantin but with slower onset and more prolonged effect. However, over 400 times more Dolantin was administered to the subject animals relative to the quantity of crotoxin. On a molar basis the difference is even more acute. Dolantin's molar equivalent with a molecular weight (M_{w}) of 283.8 was 0.140 millimoles in comparison to crotoxin's M_{w}, at a minimum of 24,000 was 0.004 micromoles, a 35,000 fold difference. With respect to acetylsalicylic acid, with a molecular weight of approximately 380, the difference is measured at over 300,000.

The data further suggested that the combination of the drugs could be beneficial giving the useful rapid onset style effectiveness displayed by Dolantin and the prolonged effect of the Crotoxin, in addition to the enhancement of the antinociceptive effect with Acetylsalicylic acid. Further benefits would be reduced dependence on a known addictive drug with significant side effects.

It will of course be understood that the invention is not limited to the specific details described herein which are given by way of example only and that various modifications and alterations are possible without departing from the scope of the invention as defined in the appended claims.

TABLE 1
Effects of Crotoxin on pain response in mice induced by Hot Plate (Mean ± SD)

| | | Doses | Time lapse before pain response (Seconds) | | | | | | |
|---|---|---|---|---|---|---|---|---|---|
| | # of | (μg/kg, | | After drug | | | | | |
| Groups | Mice | ip) | Pre-drug | 30 min | 60 min | 90 min | 120 min | 150 min | 180 min |
| Control | 10 | | 13.49 ± | 13.84 ± | 13.52 ± | 14.11 ± | 13.93 ± | 15.56 ± | 11..51 ± |
| (NS) | | | 3.32 | 4.47 | 5.12 | 4.81 | 5.04 | 4.37 | 2.14 |
| Dolantin | 10 | 40000 | 13.20 ± | 56.14 ± | 55.49 ± | 36.36 ± | 34.03 ± | — | — |
| | | | 2.65 | 10.21** | 11.94** | 16.94** | 18.47** | | |
| Crotoxin | | | | | | | | | |
| Small | 10 | 32 | 13.59 ± | — | — | 19.10 ± | 17.17 ± | 19.58 ± | 17.44 ± |
| dose | | | 4.99 | | | 8.04 | 10.00 | 14.30 | 6.92* |
| | | | | | | (35.36%) | (23.26%) | (25.84%) | (51.52%) |
| Medium | 10 | 45 | 13.77 ± | — | — | 28.74 ± | 27.89 ± | 29.48 ± | 31.70 ± |
| dose | | | 2.85 | | | 18.52* | 19.93* | 18.36* | 19.90** |
| | | | | | | (103.69%) | (100.22%) | (89.46%) | (175.41%) |
| Large | 10 | 90 | 13.72 ± | — | — | 25.25 ± | 24.50 ± | 32.21 ± | 43.42 ± |
| dose | | | 3.83 | | | 6.33** | 11.51* | 12.3** | 16.30** |
| | | | | | | (78.95%) | (75.88%) | (107.01%) | (276.91%) |

TABLE 2
The effects of crotoxin on the mouse hot plate response (X ± s)

| | | | Pain thresholds (secs) | | | |
|---|---|---|---|---|---|---|
| | | Dose | | After administration | | |
| Group | N | (μg · kg⁻¹) | Baseline | 1 | 2 | 3 h |
| Control | 10 | — | 17.51 ± 2.93 | 18.55 ± 2.38 | 17.32 ± 2.68 | 18.38 ± 4.55 |
| Acetylsalicylic acid | 10 | 400⁺ | 17.36 ± 2.72 | 28.12 ± 6.95** | 24.50 ± 14.14 | 26.22 ± 14.32 |
| Crotoxin | | | | | | |
| Large dose | 10 | 66.5 | 15.57 ± 3.14 | 24.50 ± 7.34* | 35.23 ± 18.50** | 39.95 ± 17.14** |
| Medium dose | 10 | 44.3 | 17.50 ± 3.62 | 22.90 ± 9.79 | 29.75 ± 11.72** | 29.95 ± 11.62** |
| Small dose | 10 | 29.5 | 16.62 ± 3.99 | 18.36 ± 9.60 | 20.15 ± 10.68 | 20.05 ± 7.30 |

⁺mg · kg⁻¹
*P < 0.05,
**P < 0.01 vs control group.

TABLE 3
The effects of crotoxin on the mouse writhing responses (X ± s)

| | | Dose | The number of writhings after |
|---|---|---|---|
| Group | N | (μg · kg⁻¹) | administration (times/10 min) |
| Control | 10 | | 29.40 ± 8.81 |
| Acetylsalicylic | 10 | 400⁺ | 8.80 ± 6.36** (70.07%) |
| acid | | | |
| Crotoxin | | | |
| Small dose | 10 | 29.5 | 20.40 ± 17.08 (30.61%) |
| Medium dose | 10 | 44.3 | 14.90 ± 9.37* (49.32%) |
| Large dose | 10 | 66.5 | 4.70 ± 6.63** (84.01%) |

⁺mg · kg⁻¹
*P < 0.05,
**P < 0.01 vs control group.
Percentage inhibition compared with control group is shown in parentheses.

TABLE 4
Analgesic effects of crotoxin in the rat tail-flick induced by electric
stimulation (X ± s)

| | | | Pain threshold (sec) | | | |
|---|---|---|---|---|---|---|
| | | Dose | | After administration | | |
| Group | N | (μg · kg⁻¹) | Baseline | 1 | 2 | 3 h |
| Control | 10 | 0 | 0.25 ± 0.11 | 0.29 ± 0.09 | 0.30 ± 0.07 | 0.25 ± 0.09 |
| Crotoxin | 10 | 44.3 | 0.25 ± 0.09 | 0.31 ± 0.14 | 0.43 ± 0.18* | 0.41 ± 0.19* |

*P < 0.05 vs control group.

TABLE 5
Analgesic effects of icv injection of crotoxin in acetic acid
writhing test in mice (X ± s)

| | | Dose | |
|---|---|---|---|
| Group | N | (μg · kg⁻¹) | The number of writhings (times/10 min) |
| Control | 10 | — | 20.3 ± 8.94 |
| Crotoxin | 10 | 0.3 | 9.4 ± 7.02** |

*P < 0.05,
**P < 0.01 vs control group.

TABLE 6
The effects of atropine on analgesia of crotoxin in
the mouse hot plate test (X ± s)

| | | | Pain threshold (sec) | |
|---|---|---|---|---|
| | | Dose | | After drug |
| Group | N | (mg · kg⁻¹) | Baseline | administration |
| NS + NS | 10 | 2 + 2 | 17.12 ± 2.46 | 18.59 ± 4.55 |
| NS + Atropine | 10 | 2 + 0.5 | 16.87 ± 3.10 | 18.46 ± 3.25 |
| Crotoxin + NS | 10 | 44.3^{Δ} + 2 | 16.19 ± 2.73 | 31.89 ± 12.52** |
| Crotoxin + | 10 | 44.0^{Δ} + 0.5 | 16.81 ± 3.02 | 38.22 ± 16.91** |
| Atropine | | | | |

^{Δ}μg · kg⁻¹
*P < 0.05,
**P < 0.01 vs control group.

TABLE 7
The effects of atropine on analgesia of crotoxin in
the mouse acetic acid writhing test (X ± s)

| | | | The number of writhings |
|---|---|---|---|
| Drug | N | Dose (mg · kg⁻¹) | (times/10 min) |
| NS + NS | 10 | 2 + 2 | 19.75 ± 5.06 |
| NS + Atropine | 10 | 2 + 10 | 18.60 ± 9.61 |
| Crotoxin + NS | 10 | 44.3^{Δ} + 2 | 9.33 ± 5.43** |
| Crotoxin + Atropine | 10 | 44.3^{Δ} + 10 | 10.42 ± 12.11* |

^{Δ}μg · kg⁻¹
*P < 0.05,
**P < 0.01 vs control group.

TABLE 8
The effects of naloxone on analgesia of crotoxin in the
mouse hot plate test (X ± s)

| | | | Pain threshold s | |
|---|---|---|---|---|
| | | Dose | | After drug |
| Group | N | (mg · kg⁻¹) | Baseline | administration |
| NS + NS | 10 | 2 + 2 | 16.19 ± 2.24 | 18.35 ± 4.30 |
| NS + Naloxone | 10 | 2 + 3 | 16.97 ± 3.64 | 19.04 ± 4.88 |
| Crotoxin + NS | 10 | 44.3^{Δ} + 2 | 17.73 ± 2.85 | 29.87 ± 14.45 |
| Crotoxin + Naloxone | 10 | 44.3^{Δ} + 3 | 16.68 ± 2.37 | 31.44 ± 12.56 |

^{Δ}μg · kg⁻¹
P < 0.05, P < 0.01 vs control group.

TABLE 9
The effect of acetylsalicylic acid on analgesia of crotoxin in the
mouse acetic acid writhing test (X ± s)

| | | The number of writhings |
|---|---|---|
| Drug | Dose (mg · kg⁻¹) | (times/10 min) |
| NS + NS | 2 + 2 | 28.90 ± 8.14 |
| NS + Acetylsalicylic acid | 2 + 0.3 | 12.13 ± 9.04 |
| Crotoxin + NS | 44.3^{Δ} + 2 | 17.71 ± 5.35 |
| Crotoxin + | 44.3^{Δ} + 0.3 | 5.5 ± 4.33^{#++} |
| Acetylsalicylic acid | | |

^{Δ}μg · kg⁻¹
P < 0.05, P < 0.01 vs control group.
^{#}P < 0.05 vs acetylsalicylic acid group.
⁺⁺P < 0.01 vs crotoxin group.

What is claimed is:

1. A pharmaceutical composition comprising a therapeutically effective amount of toxin from the group including crotoxin and mojavetoxin having corresponding biological activity and a pharmaceutically acceptable carrier for use in inhibiting or controlling pain.

2. The composition of claim 1 wherein the crotoxin is obtained from the snake Crotalus durissus terrificus and the mojavetoxin is obtained from the snake Crotalus scutulatus scutulatus.

3. The composition of claim 1 which further comprises a therapeutically effective amount of acetylsalicylic acid whereby the toxin and acetylsalicylic acid together produce a synergistic effect providing enhanced pain relief.

4. The composition of claim 1 for parenteral (intravenous, intramuscular or subcutaneous) administration delivering between 0.13 mcg.kg⁻¹ of body weight per day up to a maximum of 40 mcg.kg⁻¹ of body weight per day.

5. The composition of claim 1 for topical administration comprising substantially between 6 mcg and 1 mg of toxin per gram of base.

6. The composition of claim 5 in which the toxin is crotoxin at a concentration of 100-200 mcg per gram of base.

7. A method of producing and enhancing analgesia comprising administering an effective amount of toxin from the group including crotoxin and mojavetoxin having corresponding biological activity that is characterized by its ability to bind to presynaptic and postsynapic receptor sites resulting in an inhibition of aceylcholine release and its' activity.

8. A method of treatment of pain in one of the human and the animal body comprising administering an effective amount of a composition comprising a toxin from the group including crotoxin and mojavetoxin having corresponding biological activity.

9. The method of claim 7 wherein the composition includes a therapeutically effective amount of acetylsalicylic acid whereby the toxin and acetylsalicylic acid together in composition produce a synergistic effect providing enhanced pain relief.

10. The method of claim 8 wherein the composition includes a therapeutically effective amount of acetylsalicylic acid whereby the toxin and acetylsalicylic acid together in composition produce a synergistic effect providing enhanced pain relief.

11. The method of claim 5 comprising administering the toxin composition ranging from at least once every other day to several applications daily.
